SEQUENCE LISTING (29 sequences)

SEQ ID NO: 1 - protein, 12 aa, cone snail
  MOD_RES (12)..(12): AMIDATION
GCCSDPRCAWRC

SEQ ID NO: 2 - protein, 12 aa, artificial
  synthetic peptide
GCCSDPRCAWRC

SEQ ID NO: 3 - protein, 12 aa, artificial
  synthetic peptide
  MOD_RES (12)..(12): AMIDATION
GCCSDKRCAWRC

SEQ ID NO: 4 - protein, 12 aa, artificial
  synthetic peptide
GCCSDKRCAWRC

SEQ ID NO: 5 - protein, 11 aa, cone snail
  misc_feature (10)..(10): Xaa can be any naturally occurring amino acid
VCCGYKLCHXC

SEQ ID NO: 6 - protein, 11 aa, artificial
  synthetic peptide
  misc_feature (10)..(10): Xaa can be any naturally occurring amino acid
  MOD_RES (11)..(11): AMIDATION
VCCGYKLCHXC

SEQ ID NO: 7 - protein, 11 aa, artificial
  synthetic peptide
  misc_feature (10)..(10): Xaa can be any naturally occurring amino acid
VCCGYPLCHXC

SEQ ID NO: 8 - protein, 11 aa, artificial
  synthetic peptide
  misc_feature (10)..(10): Xaa can be any naturally occurring amino acid
  MOD_RES (11)..(11): AMIDATION
VCCGYPLCHXC

SEQ ID NO: 9 - protein, 16 aa, cone snail
  MOD_RES (16)..(16): AMIDATION
GCCSDPRCNMNNPDYC

SEQ ID NO: 10 - protein, 16 aa, cone snail
  MOD_RES (16)..(16): AMIDATION
GCCSLPPCAANNPDYC

SEQ ID NO: 11 - protein, 16 aa, cone snail
  MOD_RES (16)..(16)
GCCSLPPCALSNPDYC

SEQ ID NO: 12 - protein, 16 aa, cone snail
  MOD_RES (16)..(16): AMIDATION
GCCSNPVCHLEHSNLC

SEQ ID NO: 13 - protein, 18 aa, cone snail
  misc_feature (3)..(3): Xaa can be any naturally occurring amino acid
  MOD_RES (18)..(18): AMIDATION
RDXCCYHPTCNMSNPQIC

SEQ ID NO: 14 - protein, 16 aa, cone snail
  MOD_RES (16)..(16): AMIDATION
GCCSYPPCFATNSDYC

SEQ ID NO: 15 - protein, 16 aa, cone snail
  MOD_RES (16)..(16): AMIDATION
GCCSYPPCFATNSGYC

SEQ ID NO: 16 - protein, 16 aa, cone snail
  MOD_RES (16)..(16): AMIDATION
GCCSHPACAGNNQHIC

SEQ ID NO: 17 - protein, 19 aa, cone snail
  MISC_FEATURE (4)..(4): 4-carboxyglutamate
  MISC_FEATURE (16)..(16): 4-hydroxyproline
IRDXCCSNPACRVNNXHVC

SEQ ID NO: 18 - protein, 17 aa, cone snail
  MOD_RES (17)..(17): AMIDATION
GGCCSHPACAANNQDYC

SEQ ID NO: 19 - protein, 15 aa, cone snail
  MOD_RES (15)..(15): AMIDATION
GCCSYPPCFATNPDC

SEQ ID NO: 20 - protein, 15 aa, cone snail
  MOD_RES (15)..(15): AMIDATION
GCCSDPRCNYDHEIC

SEQ ID NO: 21 - protein, 12 aa, cone snail
  MOD_RES (12)..(12): AMIDATION
ACCSDRRCRWRC

SEQ ID NO: 22 - protein, 14 aa, cone snail
  MOD_RES (14)..(14): AMIDATION
GRCCHPACGKNYSC

SEQ ID NO: 23 - protein, 13 aa, cone snail
  MOD_RES (13)..(13): AMIDATION
ECCNPACGRHYSC

SEQ ID NO: 24 - protein, 15 aa, cone snail
  MOD_RES (15)..(15): AMIDATION
ECCNPACGRHYSCGK

SEQ ID NO: 25 - protein, 13 aa, cone snail
  MOD_RES (13)..(13): AMIDATION
ECCNPACGKHFSC

SEQ ID NO: 26 - protein, 13 aa, cone snail
  MOD_RES (13)..(13): AMIDATION
ICCNPACGPKYSC

SEQ ID NO: 27 - protein, 11 aa, cone snail
  MISC_FEATURE (10)..(10): 4-hydroxyproline
VCCGYKLCHXC

SEQ ID NO: 28 - protein, 12 aa, cone snail
  MISC_FEATURE (11)..(11): 4-hydroxyproline
GICCGVSFCYXC

SEQ ID NO: 29 - protein, 13 aa, cone snail
  MISC_FEATURE (12)..(12): 4-hydroxyproline
NGVCCGYKLCHXC

(19) United States
(12) Patent Application Publication
(10) Pub. No.: US 2007/0270572 A1
(43) Pub. Date: Nov. 22, 2007

(54) Conformational Switches in Toxin Folding and Uses Thereof

(75) Inventors: Kini Manjunatha, Singapore (SG); Tse Siang Kang, Singapore (SG)

Correspondence Address: CAROL NOTTENBURG, 814 32ND AVE 5, SEATTLE, WA, 98144, US

(73) Assignee: National Universitynof Singapore, Singapore (SG)

(21) Appl. No.: 11/662,507

(22) PCT Filed: Sep. 9, 2005
(86) PCT No.: PCT/SG05/00309

§ 371 Date: Mar. 8, 2007

Related U.S. Application Data
(60) Provisional application No. 60/608,151, filed on Sep. 9, 2004.

(51) Int. Cl. C07K 2/00; C07K 7/00

(52) U.S. Cl. 530/327; 530/329

(57) ABSTRACT

There is provided a method of altering the conformation of a peptide from a globular conformation to a ribbon conformation or vice versa comprising removing or introducing a conformation-inducing residue into the peptide. In particular, there is provided a method of altering the conformation of a peptide, the method comprising modifying a peptide comprising the sequence of Formula (I) to introduce a proline residue two positions N-terminal to Cys3 or to remove a proline residue that is two positions N-terminal to Cys3, wherein: Formula (I) is -Cys1-Cys2-Xₘ-Cys3-Xn-Cys4-; Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3; X is any amino acid; and m and n are the same or different and each is equal to or greater than 1.

### CROSS-REFEREMCE TO RELATED APPLICATION

This application claims benefit and priority from U.S. provisional patent application No. 60/608,151, filed on Sep. 9, 2004, the contents of which are incorporated herein by reference.

### FIELD OF INVENTION

The present invention relates generally to novel peptides, and specifically to novel peptides useful as peptide or protein scaffolds for drug design.

### BACKGROUND OF THE INVENTION

The listing or discussion of a prior-published document in this specification should not necessarily be taken as an acknowledgement that the document is part of the state of the art or is common general knowledge. All documents listed are hereby incorporated herein by reference.

Proteins play a crucial role in almost all biological processes through their specific interactions with other biomolecules. This seemingly boundless and exciting therapeutic potential of proteins has its associated disadvantages. Problems such as denaturation, poor absorption and intestinal permeability, antigenicity, difficulty in manipulation and modification, and route of administration (for example, intravenous) are seen as the major obstacles in the use of these precious macromolecules as therapeutic agents. Despite the larger size of proteins, only a small number of amino acid residues form the functional site that is involved in their interactions which is responsible for the biological properties. In vitro experiments also show that short peptides containing the functional site of the proteins exhibit the biological activity of the parent protein molecule. Complemented with the advancement of combinatorial chemistry and solid phase peptide synthesis, the importance and vast potential of utilizing peptides and proteins as therapeutic agents is rapidly gaining importance and recognition. The diverse conformational and functional possibilities that are available, serve as a valuable source of potential ligands in drug design and development. However, short linear peptides would face problems such as enzymatic digestion, as well as suffer entropic cost in binding due to its flexibility.

The recent two decades have seen the increasing focus and utilization of protein engineering to circumvent some of the problems that impede the development of proteins as drug leads. Techniques such as utilization of protein scaffolds to incorporate novel bioactive peptides, minimization of proteins to create “mini-proteins” are gradually gaining popularity.

Another important strategy utilized would be usage of small, conformationally restrained and rigid structures to incorporate novel activities. Besides conferring stability and locking the active segment in the conformationally correct structure, such strategy also minimizes antigenicity of the epitopes. One such example is cyclic proteins of US patent application US 2003/0158096. The bioactive peptide in the “mini-protein” scaffold allows rapid and efficient chemical modification, manipulation and structural characterization. Most preferred mini-protein scaffolds include proteins with a number of disulfide bridges, which confer conformational stability, as well as to impart resistance to proteolytic activity and denaturation. Toxins from the venoms of snakes, scorpions, spiders and cone snails are good sources of small disulfide-rich proteins and provide an excellent repertoire of natural protein scaffolds. In these mini protein scaffolds, disulfide bonds help in determining the folding and conformation, which have a vital role in maintaining its biological potency.

One study uses venom from a scorpion as the basis of a scaffold for holding peptide sequences in place³². This has the advantage of maintaining a peptide in structure with relatively stable activity. This scorpion scaffold construct is over 30 amino acids long and may still be prone to poor absorption, intestinal permeability and antigenicity when some peptides are used in the scaffold.

A α-conotoxin isolated from Conus geographus has been used as a scaffold to host glycoprotein D of the herpes simplex virus and found to retain some antigenic properties of the native viral peptide.

### OBJECTS OF THE INVENTION

The findings of this work relate to the identification of key structural determinants responsible for the folding of α-conotoxin ImI.

Here we describe the contribution of proline in the first intercysteine loop, as well as the conserved carboxyl terminal amidation, as the major structural determinants in the folding of a class of short peptide toxins, α-conotoxins. Identification of these structural switches are useful in the design of mini protein in the desired conformation.

α-conotoxins are short, disulfide-rich peptides derived from the venom of the marine predatory cone snails. One of the key structural features of these toxins is the presence of a highly conserved cysteine framework made up of two disulfide bridges amidst its short sequence of 11-19 amino acid residues. Native α-conotoxins have a “Globular” conformation held in place with two disulfide bonds. In spite of the relatively diverse range of possible amino acid variation within the two intercysteine loops, α-conotoxins show a preference to the “Globular” conformation (C₁₋₃, C₂₋₄) over the flatter “Ribbon” (C₁₋₄, C₂₋₃) or the flexible “Beaded” (C₁₋₂, C₃₋₄) conformation. Recently, a new group of conotoxins was discovered: λ-conotoxins (or χ-conotoxin)^{2,30-31}. Though the χ/λ-conotoxins possess identical conserved quadruple cysteines in its framework, the native conformation observed was the ribbon (C₁₋₄, C₂₋₃) conformation instead of the usual globular structure seen in α-conotoxins.

In vivo assays with native globular α-conotoxin GI showed that the beaded isoform suffered a ten fold reduction in biological activity, while force-folding into the ribbon conformation abolished all nACHR antagonistic activity!¹ Conversely, χ/λ-conotoxin CMrVIA in its native ribbon conformation has a potency that is 3 orders magnitude higher as compared to the non-native globular conformation in seizure induction.² These findings emphasize the point that structural conformation has a crucial role to play in determining the biological potency of these short peptides. However, the structural features attributing to this change in disulfide linkages and conformation change are still unclear.

By synthesizing variants of a native a-conotoxin, we have shown that the C-terminal amidation and Proline residue in the 1^{st} intercysteine loop can effect a shift of the folding tendency of α-conotoxin from the native globular conformation, to the non-native ribbon conformation. By understanding the folding nature of this highly compact and stable structure, it is possible to manipulate the peptide backbone as a scaffold for insertion of short, active sequences, useful in the development of novel bioactive peptides.

### SUMMARY OF INVENTION

In one aspect, the invention provides a method of altering a protein conformation by removing, for example by deletion or substitution, one or more conformation-inducing amino acids.

In one aspect the invention provides a method of altering the conformation of a protein or a peptide from a globular conformation to a ribbon conformation comprising removing, for example by deletion or by substitution, a specific conformation-inducing residue from the protein or peptide. In one embodiment, the conformation-inducing residue is proline. In one particular embodiment, the conformation-inducing residue is proline located in a loop of a domain of the protein or peptide, for example an inter-cysteine loop of a domain defined by one or more pairs of cysteine residues forming disulfide bonds. Furthermore, an N-terminal or C-terminal cap may be added or removed at the relevant end of the protein or peptide to further promote or stabilize an induced conformational shift.

In a further aspect the invention provides a method of altering the conformation of a protein or a peptide from a ribbon conformation to a globular conformation comprising introducing, for example by insertion or by substitution, a specific conformation-inducing residue from the protein or peptide. In one embodiment, the conformation-inducing residue is proline. In one particular embodiment, the conformation-inducing residue is proline and is introduced into a loop of a domain of the protein or peptide, for example an inter-cysteine loop of a domain defined by one or more pairs of cysteine residues forming disulfide bonds. As in the previous method, an N-terminal or C-terminal cap may be added or removed at the relevant end of the protein or peptide to further promote or stabilize an induced conformational shift.

In another aspect, the invention provides a method of altering the conformation of a peptide, the method comprising modifying a peptide comprising the sequence of Formula I to introduce a proline residue two positions N-terminal to Cys3 or to remove a proline residue that is two positions N-terminal to Cys3, wherein: Formula I is -Cys1-Cys2-Xₘ-Cys3-Xₙ-Cys4-; Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3; X is any amino acid; and m and n are the same or different and each is equal to or greater than 1. In certain embodiments, the peptide has a C-terminal group that is either of a carboxy group or an amide group, and the method further includes converting the C-terminal group to the other of the carboxy group or the amide group.

In another aspect, the invention provides a method of altering the conformation of a peptide, the method comprising modifying a peptide comprising the sequence of Formula I and a C-terminal group that is either of a carboxy group or an amide group to convert the C-terminal group to the other of the carboxy group or the amide group, wherein: Formula I is -Cys1-Cys2-Xₘ-Cys3-Xₙ-Cys4-; Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3; X is any amino acid; and m and n are the same or different and each is equal to or greater than 1. In certain embodiments the method further includes introducing a proline residue two positions N-terminal to Cys3, for example by insertion or substitution, or removing a proline residue that is two positions N-terminal to Cys3.

In another aspect the invention provides a peptide comprising a conotoxin consensus sequence as defined in Formula I, and having one or more amino acid residues inserted or substituted between Cys2 and Cys3 such that the region defined by Xₘ differs from the corresponding region in any wildtype conotoxin sequence, or having one or more amino acid residues inserted or substituted between Cys3 and Cys4 such that the region defined by Xₙ differs from the corresponding region in any wildtype conotoxin sequence, wherein: Formula I is -Cys1-Cys2-Xₘ-Cys3-Xₙ-Cys4-; Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3; X is any amino acid; and m and n are the same or different and each is equal to or greater than 1. In one embodiment the peptide has a proline residue two positions N-terminal to Cys3 and a C-terminal amide group, and the peptide has the tendency to adopt a globular conformation. In another embodiment, the peptide is lacking a proline residue two positions N-terminal to Cys3 and a C-terminal carboxy group, and has the tendency to adopt a ribbon conformation. In different embodiments, the sequence RGD or RGDW is inserted between Cys2 and Cys3 or between Cys3 and Cys4.

In a further aspect the invention provides a peptide comprising the sequence as set forth in any one of SEQ ID NOS. 2, 3, 4, 6, 7 or 8.

In still a further aspect, the invention provides a peptide consisting of the sequence as set forth in any one of SEQ ID NOS. 2, 3, 4, 6, 7 or 8.

By comparing the amino acid sequences of α-conotoxins and χ/λ-conotoxins (Table 1), several differences are apparent. Firstly, unlike a-conotoxins in which the conformationally constraining proline residue is invariably present in intercysteine loop 1, χ/λ-conotoxins has a hydroxyproline residue in intercysteine loop 2 but lacks the kink-inducing residue in the first loop. Secondly, it can also be seen that the C-terminus amidation is conserved in all known α-conotoxins (except GID α-conotoxin), but consistently absent in all the 3 currently known members of the χ/λ-conotoxins. It is with these differences in mind that the synthetic peptide variants were designed.

Aside from the fact that α-conotoxin ImI is one of the most studied α-conotoxin³⁻¹⁶, ImI conotoxin was selected as a model for our investigation due to the fact that the intercysteine loop sizes are the closest to that of χ/λ-conotoxins, and that ImI conotoxin does not possess any form of post-translational modification other than the conserved C-terminal amidation. Further, the 3-dimensional structure of the native peptide, along with several of its point mutation variants had already been solved by NMR spectrometry. In this work, the following synthetic variants were designed to examine the role of proline in both intercysteine loop 1 as well as the effect of C-terminal amidation:

ImI Conotoxin:

[SEQ ID NO: 1]

Gly-Cys-Cys-Ser-Asp-Pro-Arg-Cys-Ala-Trp-Arg-Cys-
CONH₂
ImI Acid:

[SEQ ID NO: 2]

Gly-Cys-Cys-Ser-Asp-Pro-Arg-Cys-Ala-Trp-Arg-Cys-
COOH
P6K Amide:

[SEQ ID NO: 3]

Gly-Cys-Cys-Ser-Asp-Lys-Arg-Cys-Ala-Trp-Arg-Cys-
CONH₂
P6K Acid:

[SEQ ID NO: 4]

Gly-Cys-Cys-Ser-Asp-Lys-Arg-Cys-Ala-Trp-Arg-Cys-
COOH
CMrVIA Acid:

[SEQ ID NO: 5]

Val-Cys-Cys-Gly-Tyr-Lys-Leu-Cys-His-Hyp-Cys-COOH
CMrVIA Amide:

[SEQ ID NO: 6]

Val-Cys-Cys-Gly-Tyr-Lys-Leu-Cys-His-Hyp-Cys-CONH₂
CMrVIA K6P Acid:

[SEQ ID NO: 7]

Val-Cys-Cys-Gly-Tyr-Pro-Leu-Cys-His-Hyp-Cys-COOH
CMrVIA K6P Amide:

[SEQ ID NO: 8]

Val-Cys-Cys-Gly-Tyr-Pro-Leu-Cys-His-Hyp-Cys-CONH₂

### BRIEF DESCRIPTION OF THE DRAWINGS

TABLE 1: Sequence alignment of α-conotoxins and χ/λ-conotoxins. (m/n) refers to the number of residues in the 1^{st} and 2^{nd} intercysteine loops respectively, when the peptides adopt either the globular or ribbon conformation.

FIG. 1: (A) Purification of synthetic ImI Acid variant, (B) P6K Acid variant, and (C) P6K amide variant on a Phenomenex Jupiter C18 5 μp 300 Å, 250 mm×10 mm semi-preparative column, using 0.1% TFA (Eluent A) and an increasing gradient of 80% Acetonitrile with 0.1% TFA (Eluent B).

FIG. 2: (A) Oxidation profile of the various purified peptides in 100 mM Tris-HCl, 2 mM EDTA, pH 8.5. Chromatographic separation of the oxidized samples revealed 3 isoforms in each of the variants. Predominant isoforms in each variant are marked with (*).

Table 2: Air Oxidation of synthetic peptide variants. All variants oxidized into 3 possible conformers of varying proportions.

FIG. 3: Chromatographic profiling of forced-folded conformations of peptide variants. The retention time of the forced folded conformation were compared and matched with the dominant isoform derived from air oxidation.

FIG. 4: 1-Dimensional NMR spectroscopy comparing the spectrums of the (A) P6K Acid variant peak 1 with the forced-folded ribbon conformation, (B) P6K Amide variant peak 1 with the forced-folded ribbon conformation, and (C) ImI Acid with the forced-folded ribbonr conformation, (D) ImI Conotoxin with the forced-folded globular conformation, (E) CMrVIA Acid with the forced-folded ribbon conformation, (F) CMrVIA Amide with the forced-folded ribbon conformation, (G) CMrVIA K6P Acid with the forced-folded globular conformation, (H) CMrVIA K6P Amide with the forced-folded globular conformation.

FIG. 5: Mass Spectrometry profiles of the various reduced and oxidized 1ml-conotoxin and CMrVIA conotoxin variants.

TABLE 3: Mass Spectrometry summary table for the theoretical and observed mass for the peptide variants.

FIG. 6: 2-Dimensional NMR summary chart comprising of 70 ms TOCSY αH-NH region (top) and 300 ms ROESY region (bottom) defining the various spin systems and sequential connectivities. 2-D NMR experiments were carried out on the dominant structural isoform for each variant, and the samples were dissolved in 90% H₂O and 10% D₂O, pH 3.0-3.1 on Bruker DRX-500 MHz spectrometer. (A) ImI Acid Variant Peak 1, (B) P6K Acid Peak 2, (C) P6K Amide Peak 1, (D) ImI conotoxin Peak 3, (E) CMrVIA Acid Peak 3, (F) CMrVIA Amide Peak 3, (G) CMrVIA K6P Acid Peak 1, and (H) CMrVIA K6P Amide Peak 1.

TABLE 4: Chemical shifts summary for (A) ImI Acid Peak 1, (B) P6K Acid Peak 2, (C) ImI Conotoxin Peak 3, and (D) P6K Amide Peak 1.

FIG. 7: Structural modeling ImI Acid variant Peak 1 and P6K Acid variant Peak 2 performed with Accelrys Insightil molecular modeling software. Backbone RMSD for the 2 structures were 0.38±0.06 and 0.72±0.12 respectively. 3- Dimensional structure of solution structure of ImI conotoxin was obtained from Protein Data Bank.

FIG. 8: Profiles of the 2 constructs RGD in the first cystine loop (RGD1) 7a and RGD in the second intercystine loop (RGD2) 7b oxidized into 3 possible conformers of varying proportions and the ability of these to inhibit platelet aggregation of these conformers.

### DETAILED DESCRIPTION OF THE INVENTION

Peptide synthesis:

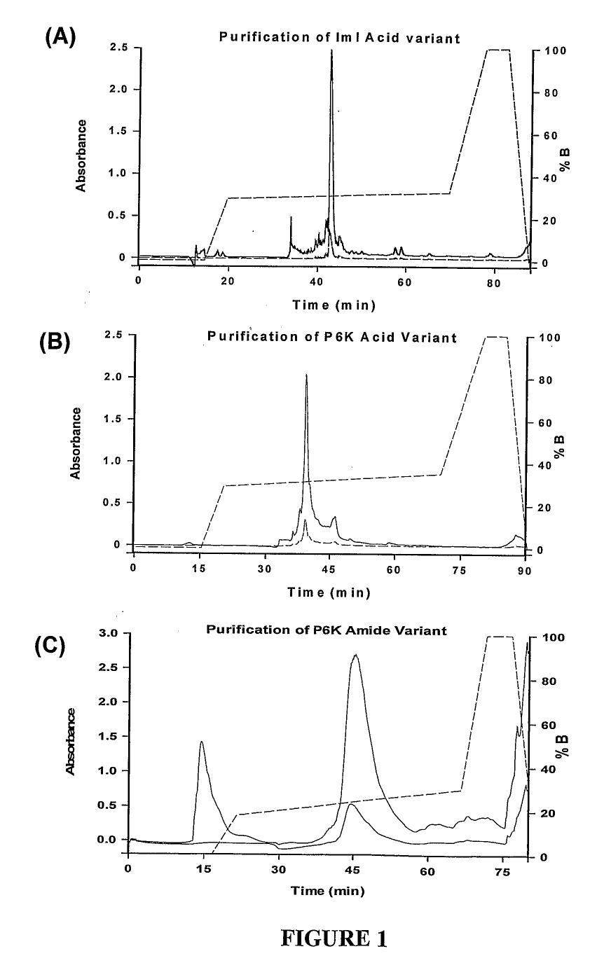

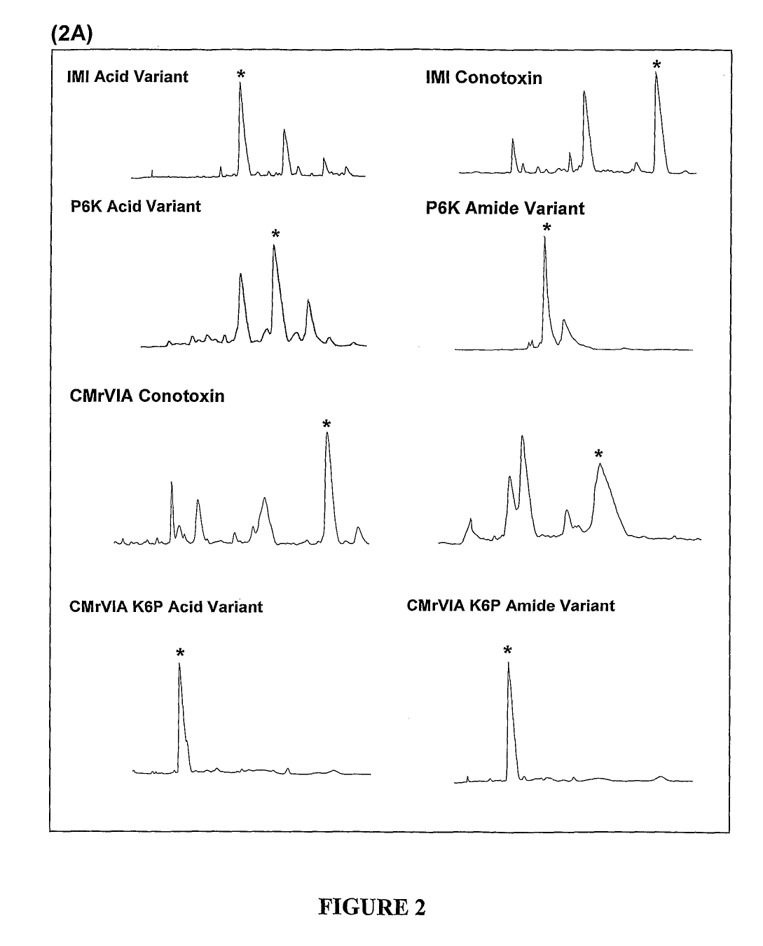

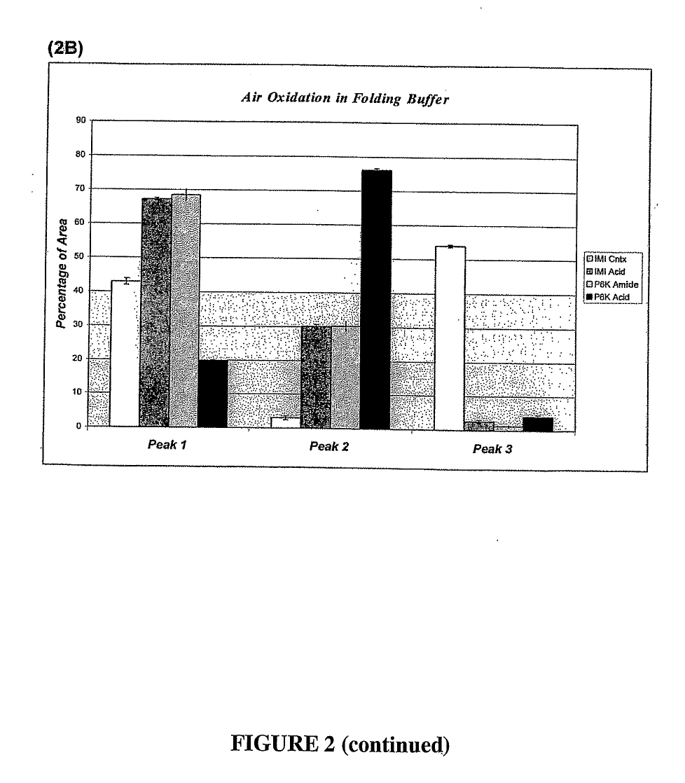

The peptide variants were synthesized by solid phase peptide synthesis with Fmoc chemistry on ABI Pioneer Model 433A Peptide Synthesizer. The amino acid residues were coupled using N-[(dimethylamino)-1H-1,2,3-triazolo[4,5-b]pyridine-1-ylmethylene]-N-methylmethanaminium hexafluorophosphate N-oxide/N,N-Diisopropylethylamine in situ neutralization chemistry. The synthetic peptides having C-terminal amidation were synthesized using Fmoc-PAL-PS support, while variants possessing a free carboxyl terminal were assembled on a pre-loaded Fmoc-L-Cys(Trt)-PEG-PS (Polyethylene glycol-polystyrene) support resin. All four cysteines in the sequences were protected by Trifluoroacetic acid (TFA)-labile Trityl group, with no selective deprotection. The synthesized peptide was then cleaved off the resin, with the concomitant removal of side chain protection groups using Trifluoroacetic acid: Ethane-dithiol: Thioanisole: Water (92.5:2.5:2.5:2.5). The crude peptides were subsequently purified by reverse-phase HPLC (FIG. 1). Purified reduced ImI conotoxin was custom ordered from Synpep Corporation (Dublin, Calif.). The purified peptides were then characterized by their molecular mass (FIG. 2). Air oxidation of the purified peptide was carried out in 100 mM Tris-HCl with 2 mM EDTA, pH 8.5, and allowed to stir in air for 48 Hr.

Isoforms within the oxidized peptide samples were then separated using reverse phase HPLC on a BioCAD SPRINT chromatographic workstation or ÄKTA™ purifier system, using a gradient of 80% acetonitrile, with either 0.1% TFA or formic acid as ion-pairing agent over 100 min on Vydac 201 SP501 C18 4.6 mm×250 mm analytical column (FIG. 2A).

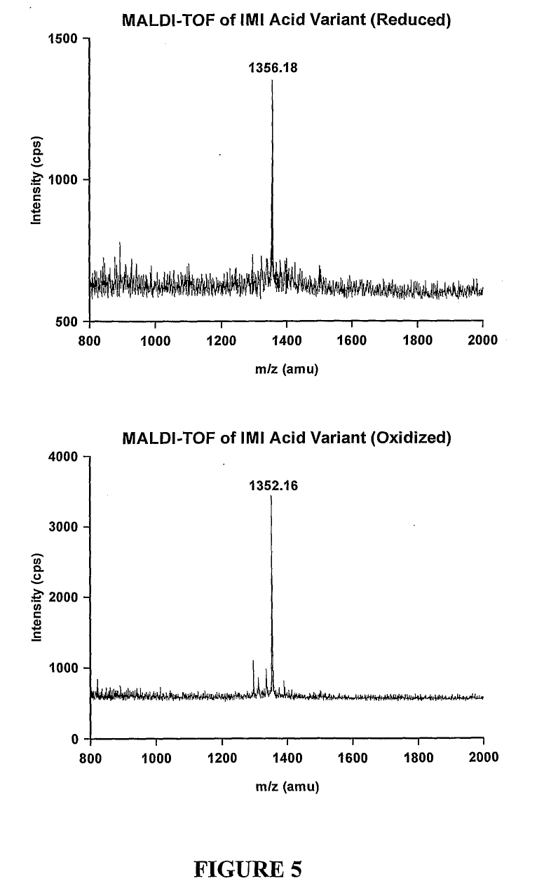

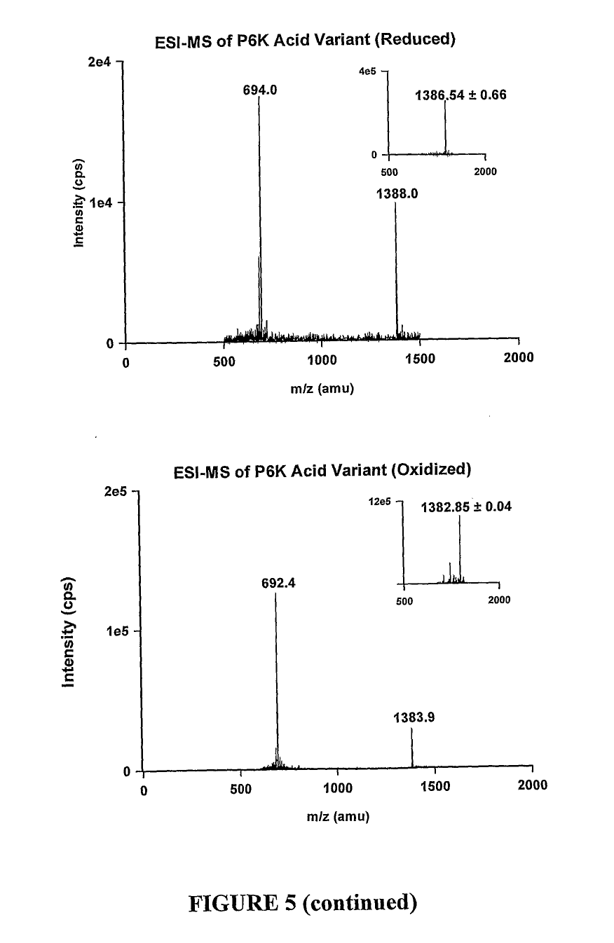

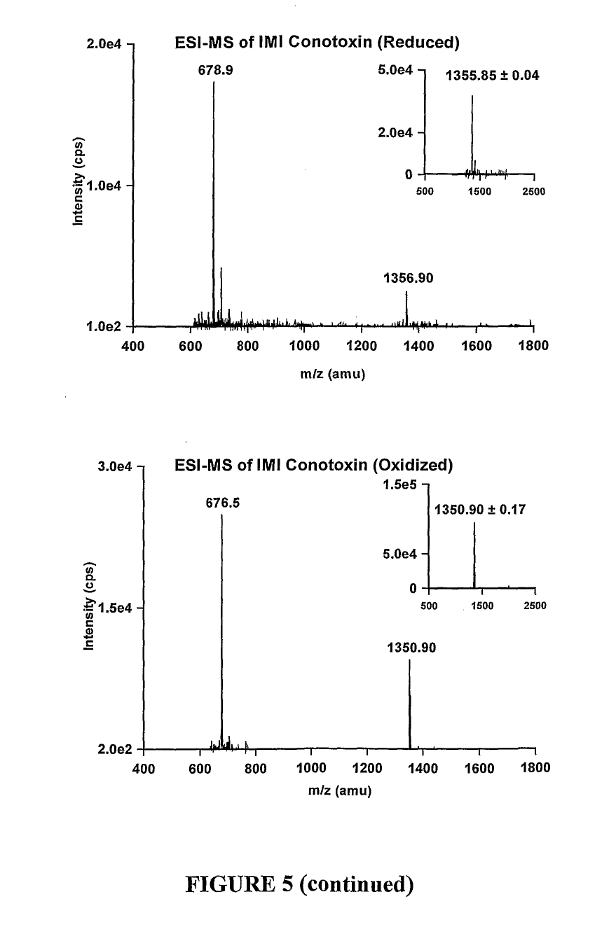

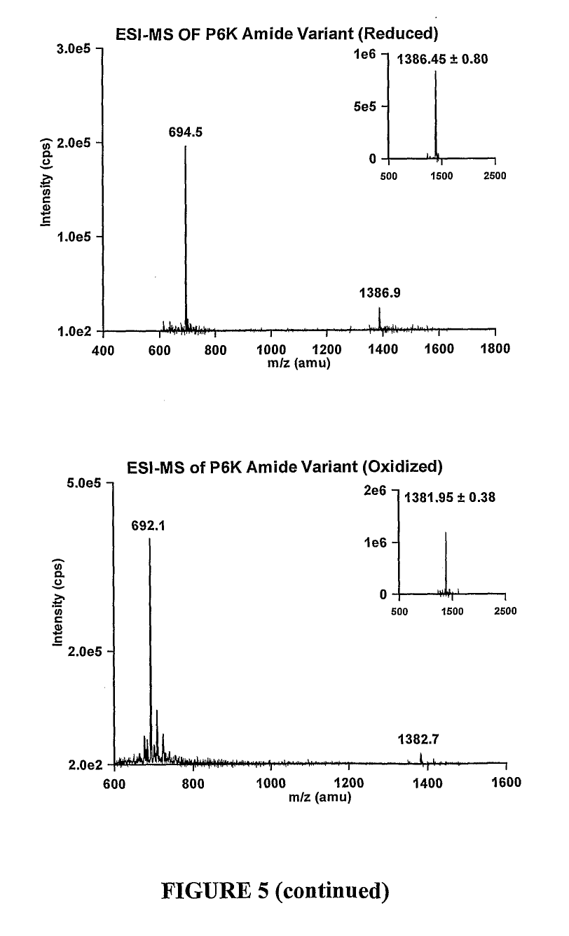

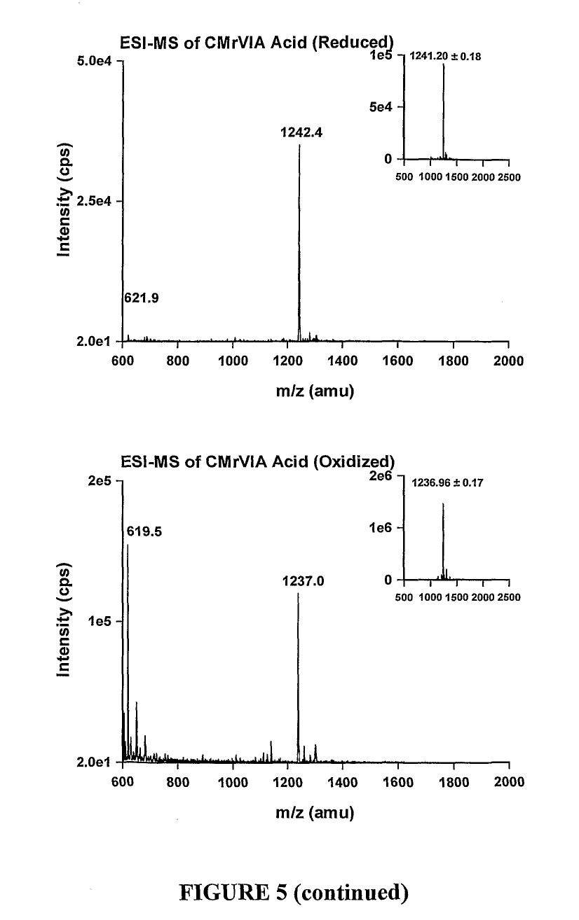

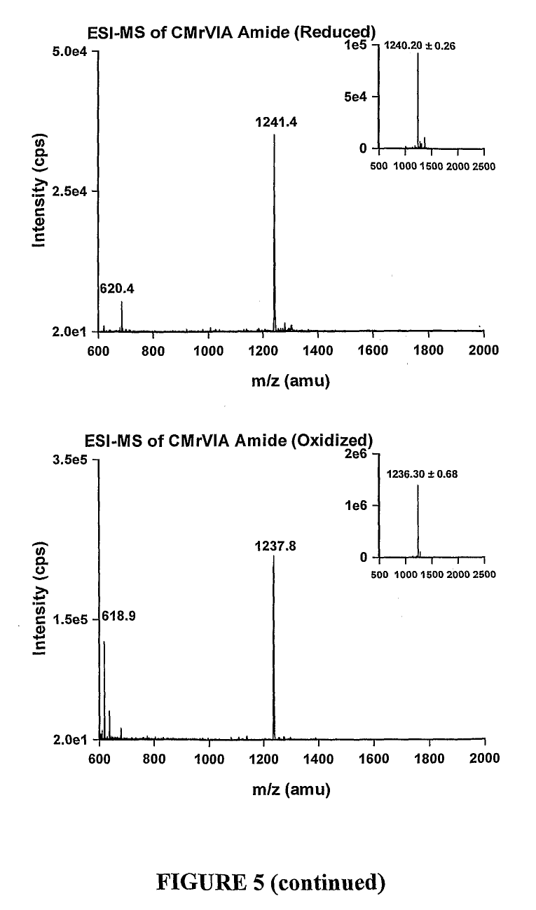

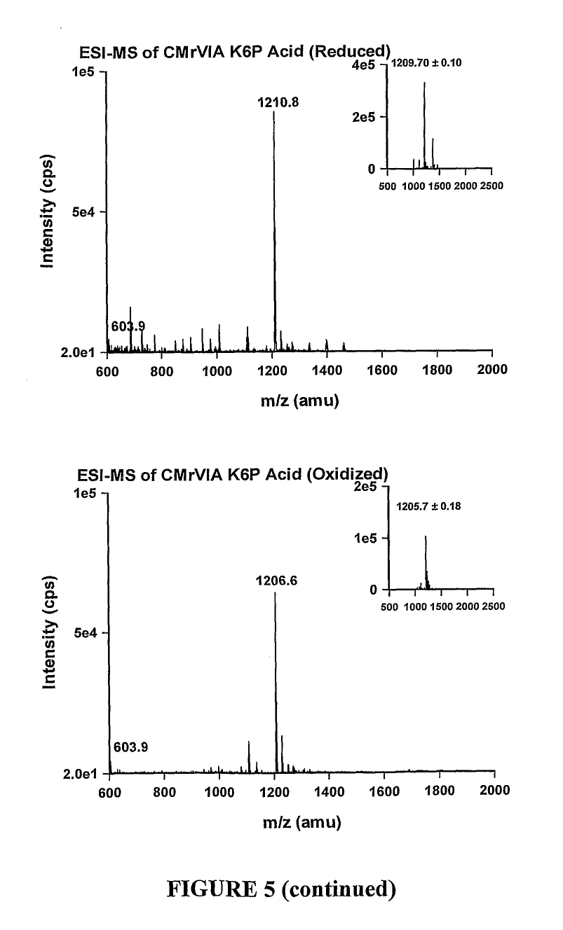

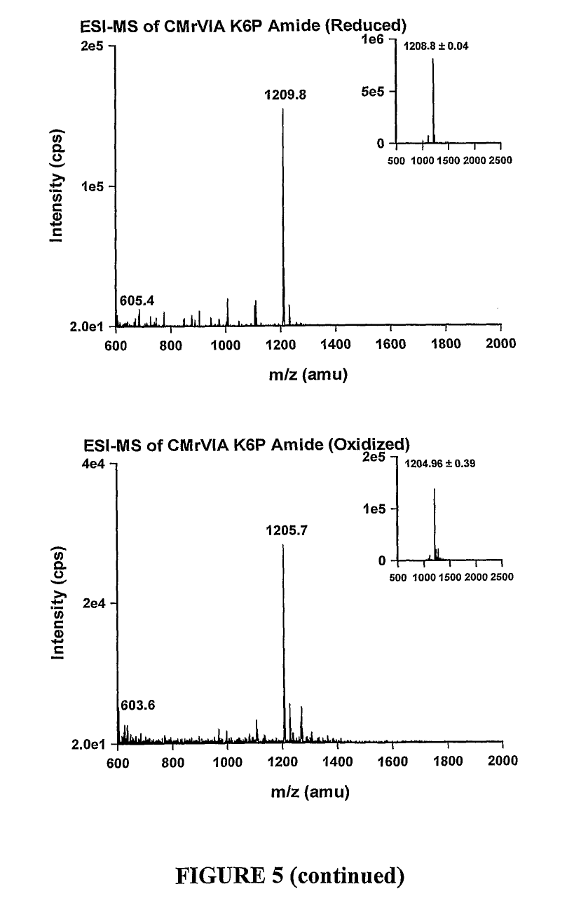

The peptides were verified to be fully reduced based on ESI-MS (Perkin Elmer Sciex API III Triple-stage Quadrupole System) prior to oxidation studies. For air oxidation, 0.1 mM of peptide was dissolved in folding buffer comprised of 100 mM Tris-CL and 2 mM EDTA, adjusted to pH 8.5, and allowed to stir in air for 48 hrs. Oxidation studies were also repeated in denaturant condition, as well as glutathione redox system (data not shown). Complete oxidation of the peptide was verified by the reduction of four mass units, which is attributed to the formation of the two disulfide bridges within the peptide backbone (FIG. 5, Table 3). Each of the synthetic variant folded into three isoforms upon oxidation (FIG. 2A, 2B, Table 2).

Iodine Oxidation

Peptide variants with the desired forced-folded disulfide linkage of choice were generated by means of selective deprotection. This involves the orthogonal side chain protection of the four cysteine residues so as to generate specific cysteine pairing of choice in the formation of the two disulfide bridges. Cysteine pairs involved in the formation of the first, and second disulfide bridge were protected using S-trityl and S-acetamidomethyl protection groups respectively. The S-trityl group which is removed during the cleavage step allows the first disulfide bond to be formed by stirring in air in 0.1 M ammonium bicarbonate (pH 8.5) at a concentration of 0.1 mg/ ml for 48 Hr.

The second pair of cysteines was deprotected and concomitantly oxidized using iodine oxidation. This was achieved by adding 0.1 M Iodine to a deaerated solution containing 0.1 mM peptide (10 equivalent/ACM) in Acetonitrile/TFA/Water (20:2:78% v/v), and stirred vigorously under nitrogen blanket for 1 min before quenching with 1 M ascorbic acid drop-wise until the solution becomes colorless. The oxidized peptide was then isolated using RP-HPLC.

Identification of Dominant Isoform from Air Oxidation

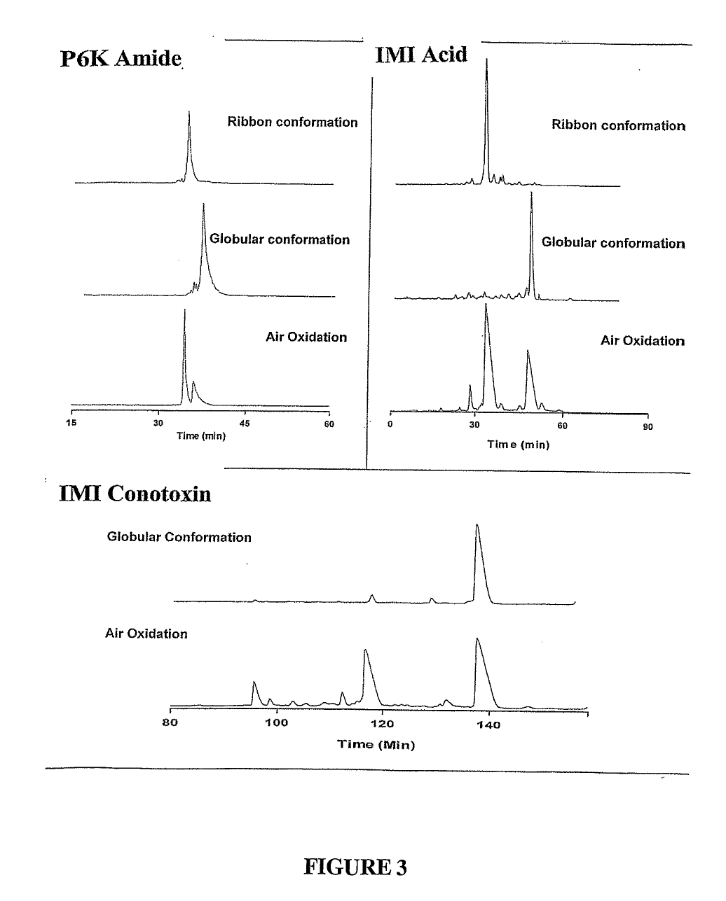

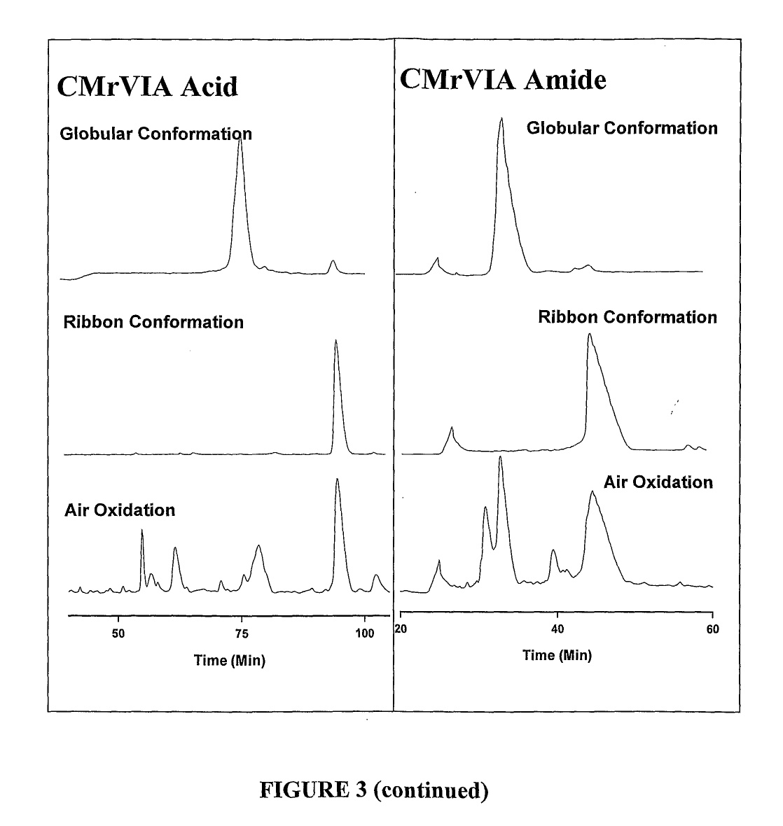

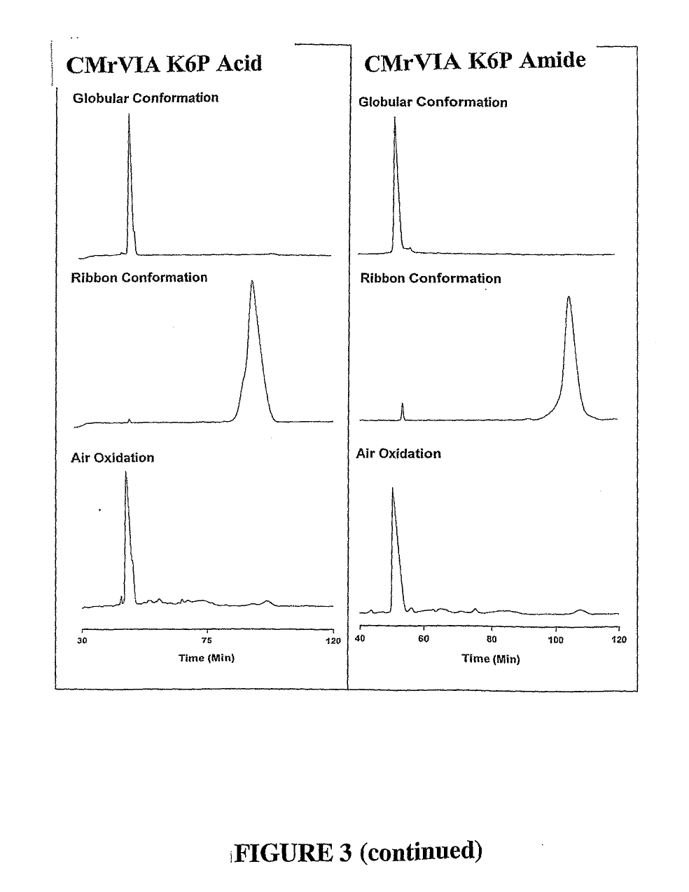

The retention time of the forced-folded conformation for the various peptide variants were compared with the corresponding air oxidation chromatographic profiles so as to identify the conformation of the dominant isoform in each variant (FIG. 3). Air oxidation of ImI conotoxin was used as a control to verify that the folding conditions used maintained the folding bias of native a-conotoxins. From the chromatographic profiling of synthetic ImI conotoxin, ImI Acid variant, P6K Amide variant, and P6K Acid variant, it can be seen that only ImI conotoxin maintained the folding bias of having globular conformation as the dominant isoform, while the other 3 variants has shifted towards the ribbon conformation (FIG. 2B, Table 3).

NMR Data:

The dominant isoform from the air oxidation studies for each variant was then analyzed on the Bruker 300 MHz spectrometer to acquire the 1-Dimensional NMR spectrum. The 1-D NMR spectrum was then compared with the spectrum of the various possible conformation obtained by selective deprotection. The conclusions obtained from the 1-D NMR analysis matches with the data of the conformation obtained using HPLC.

The three dimensional structure for the major isoform of each variant was then solved with 2-Dimensional Nuclear Magnetic Resonance Spectroscopy (FIG. 5, Table 4). In all four cases, ˜1 mM of the peptide gave NMR spectra of adequate quality for TOCSY and ROESY 2-D NMR experiments at pH 3.1 in 10% D₂O, 90%, acquired on Bruker DRX-500 MHz spectrometer. Spectra were acquired at 298 K with water suppression. TOCSY mixing time was set at 70 ms and ROESY spin-lock time of 300 ms.

Structural modeling was performed using Accelrys InsightII software with NOE constraints derived from the NMR spectrum (FIG. 5). NOE constraints were classified as Strong (1.9-3.1 Å), Medium (1.9-3.8 Å), and Weak (1.9-5.5 Å). Pseudo-atom corrections were made for methyl and methylene protons according to Wuthrich et al.¹⁷ High temperature molecular dynamics was first performed using Insightil Discover module at 300 K and 600 K at 10 ps, followed by 900 K at 20 ps. Dynamics was subsequently done at decreasing temperatures from 900 K to 400 K in steps of 100 K before cooling to 300 K by “soaking” in an assembly of water molecules at 20 ps. The 15 frames with the lowest energy levels were then overlaid with an averaged structure from 211 frames. Overlaid ImI conotoxin Peak 1 gave a backbone RMSD of 0.39±0.12. Overlaid P6K Acid Peak 2 gave a backbone RMSD of 0.51±0.09., both adopting a “Ribbon” (C₁₋₄, C₂₋₃) conformation. 2-D NMR TOCSY spectrum gave a spectrum similar to that reported by David Craik et al⁵.

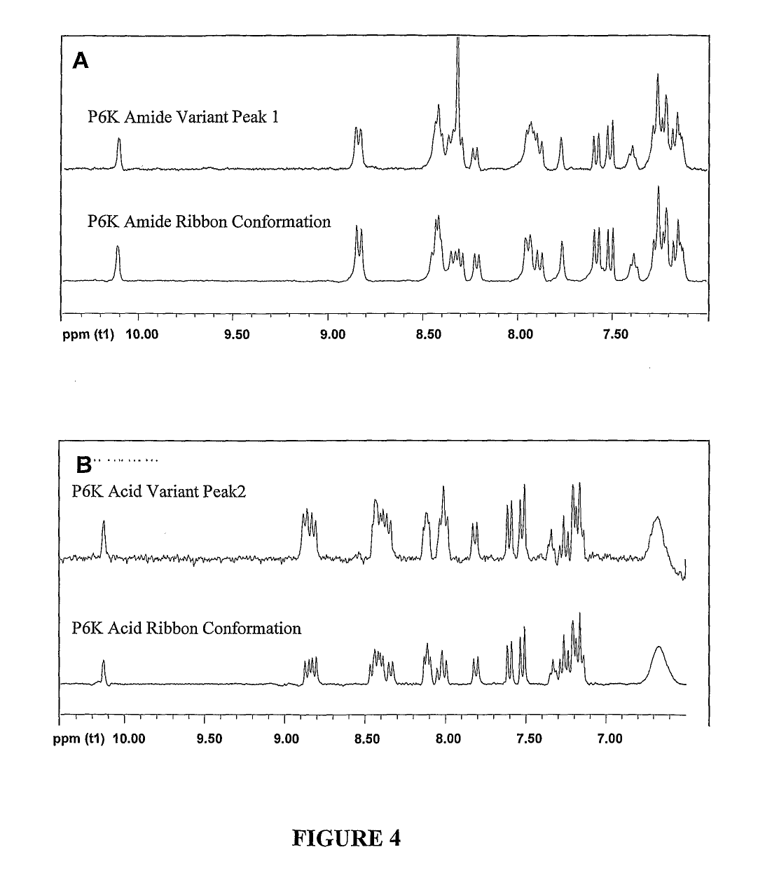

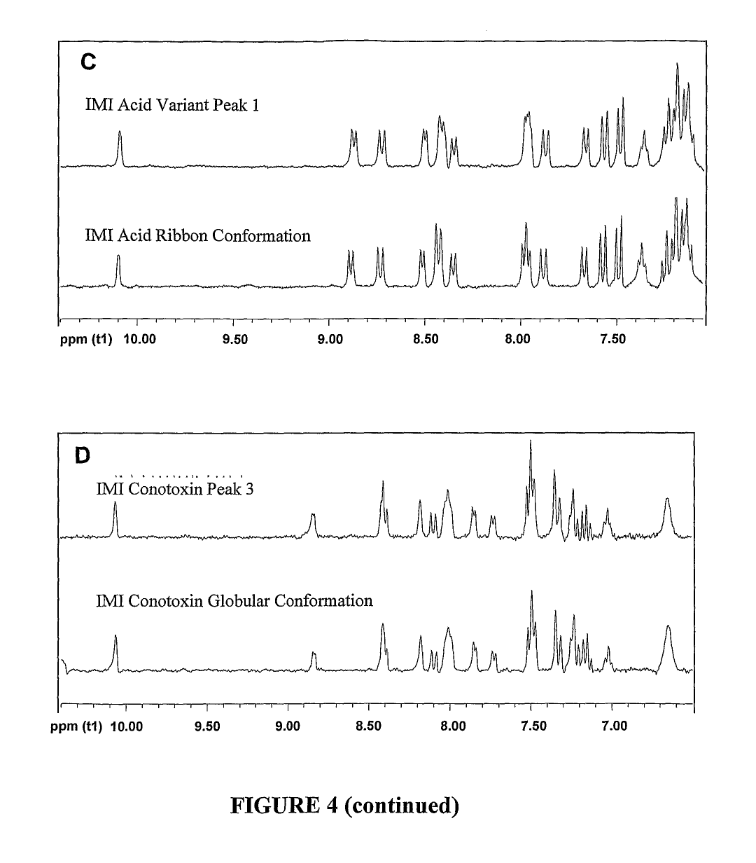

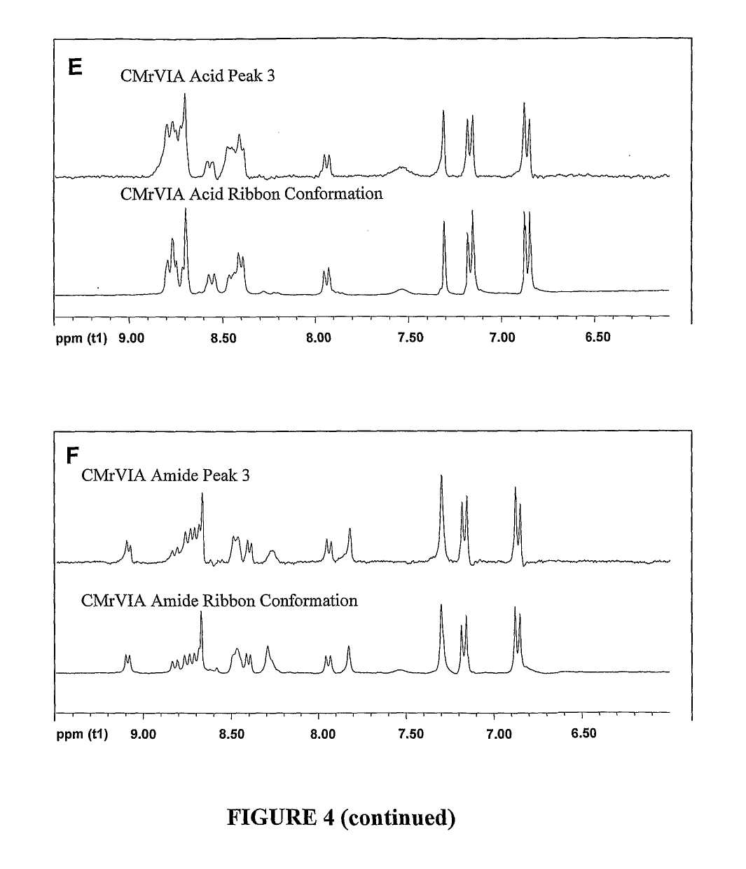

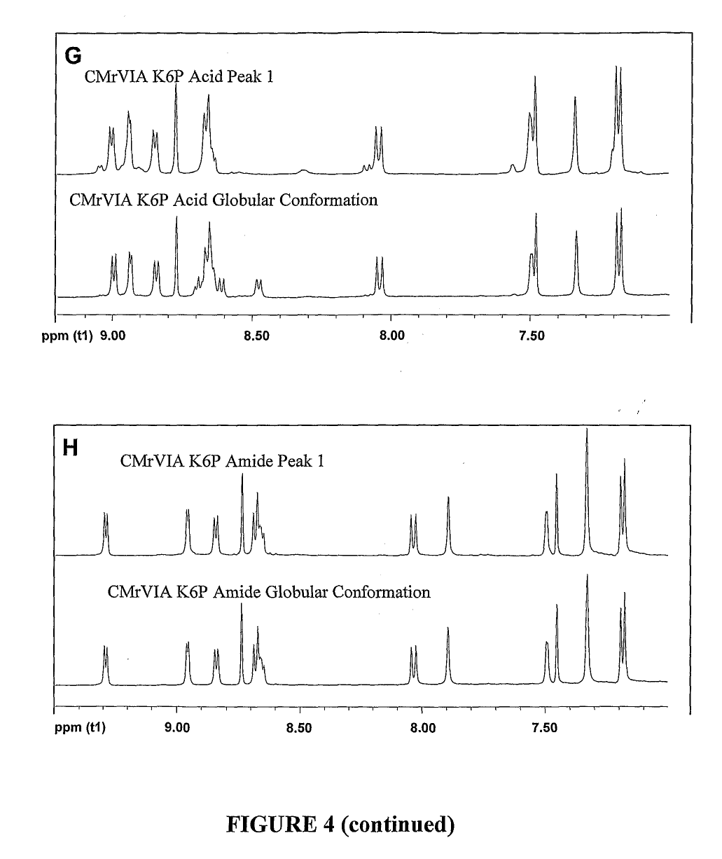

FIG. 4 demonstrates 1-Dimensional NMR spectroscopy comparing the spectrums of the P6K Acid variant peak 1 with the forced-folded ribbon conformation, P6K Amide variant peak 1 with the forced-folded ribbon conformation, and ImI Acid with the forced-folded ribbonr conformation, ImI Conotoxin with the forced-folded globular conformation, CMrVIA Acid with the forced-folded ribbon conformation, CMrVIA Amide with the forced-folded ribbon conformation, CMrVIA K6P Acid with the forced-folded globular conformation, CMrVIA K6P Amide with the forced-folded globular conformation.

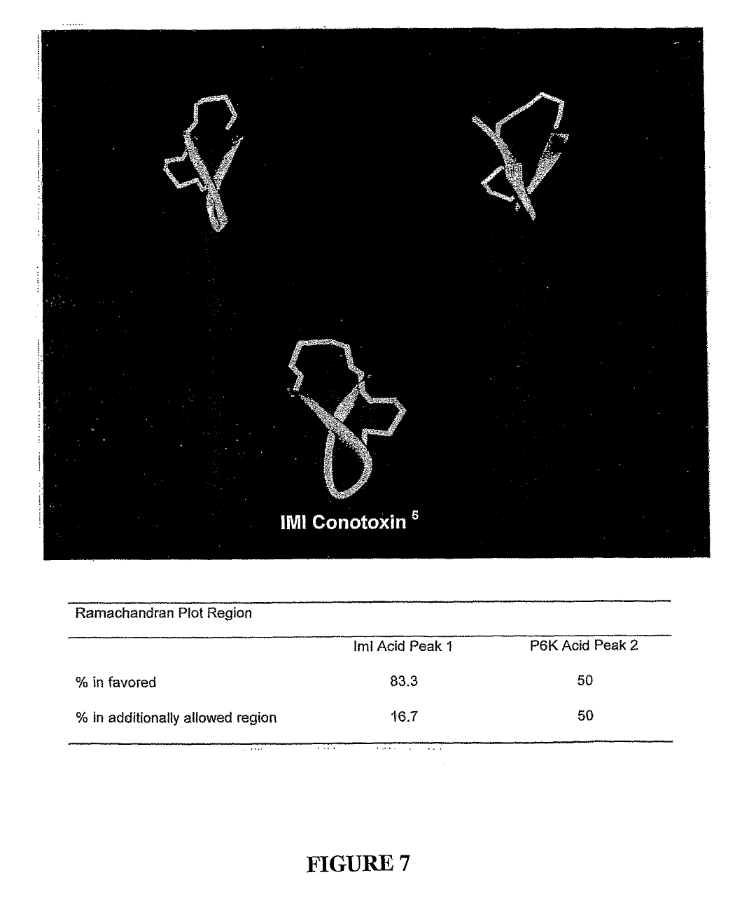

FIG. 7 shows structural modeling of ImI Acid variant Peak 1 and P6K Acid variant Peak 2 performed with Accelrys InsightII molecular modeling software and compared with solution structure of ImI conotoxin. Backbone RMSD for the 2 structures were 0.38±0.06 and 0.72±0.12 respectively. 3-Dimensional structure of solution structure of ImI conotoxin was obtained from Protein Data Bank.

Discussion

Analysis of the sequences of α-conotoxin ImI and the χ/λ-conotoxins revealed the structural differences which formed the basis to the design of the synthetic variants.

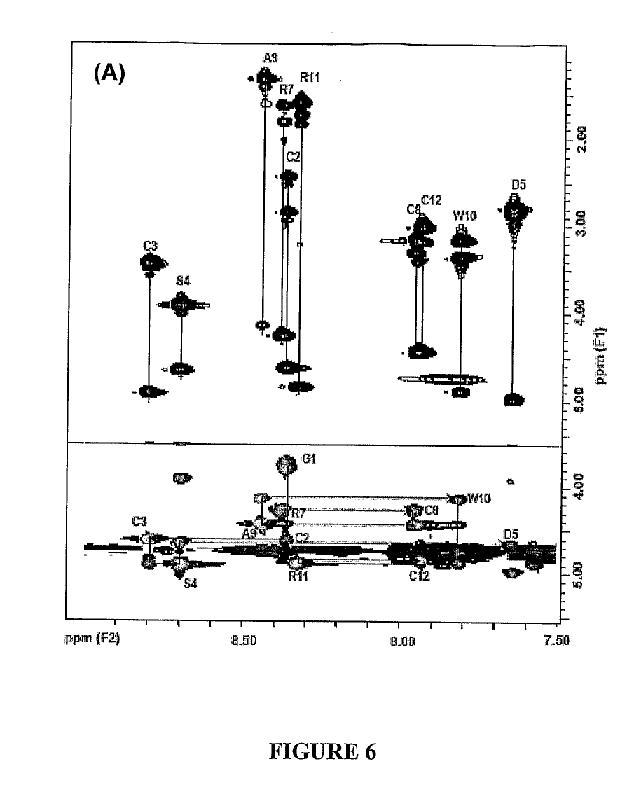

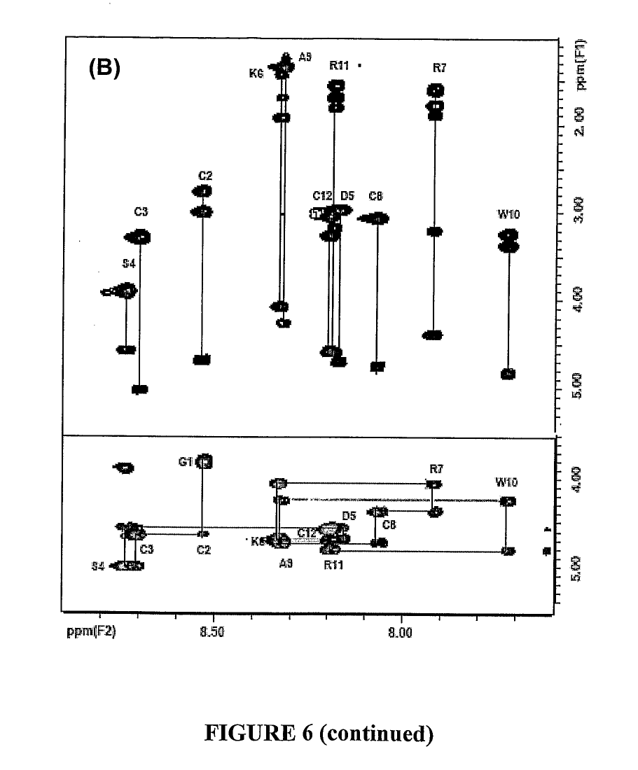

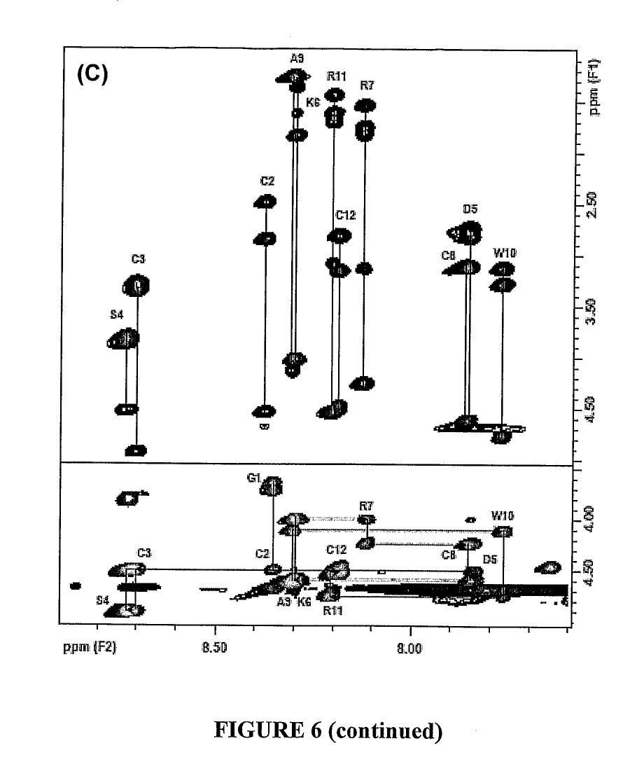

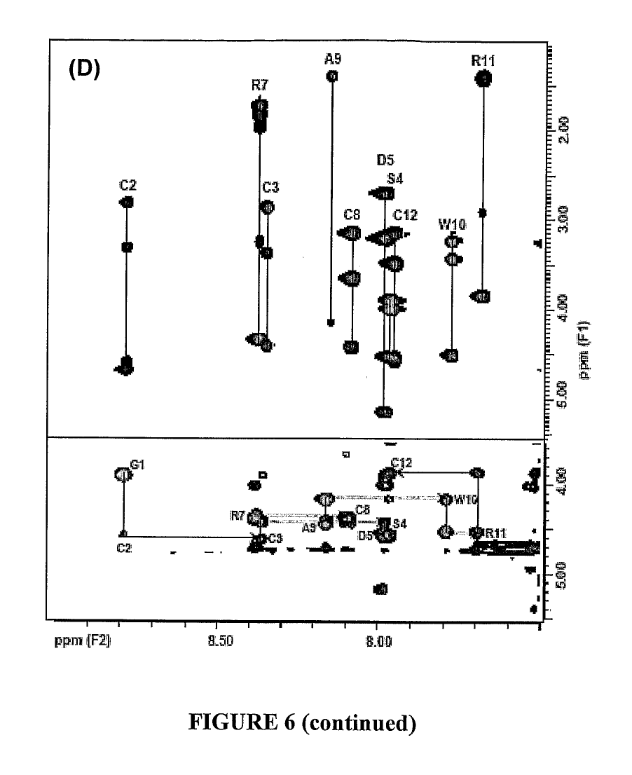

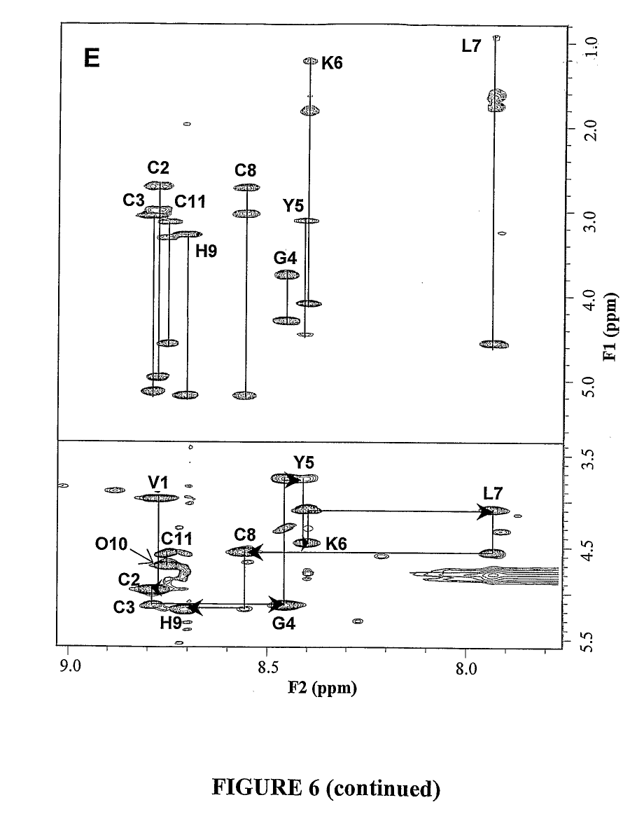

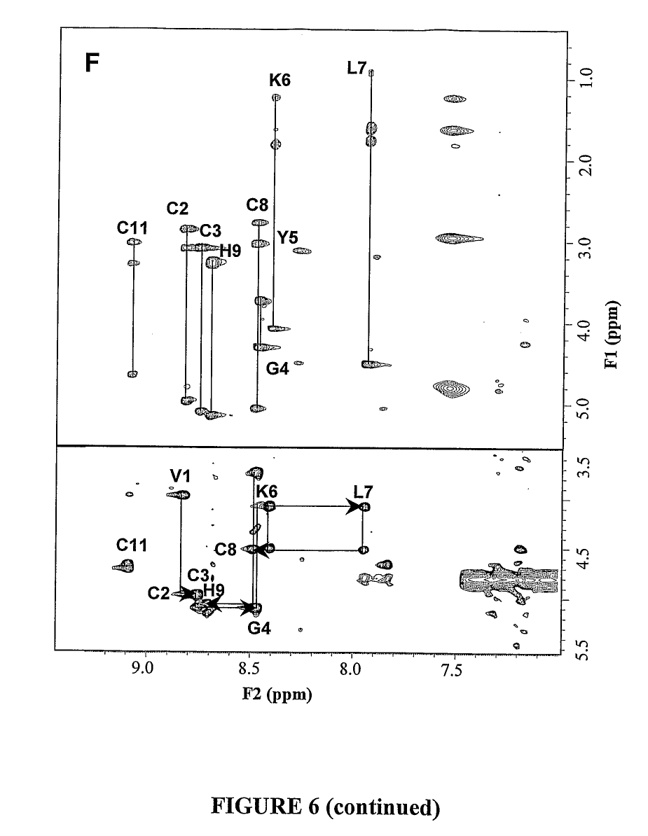

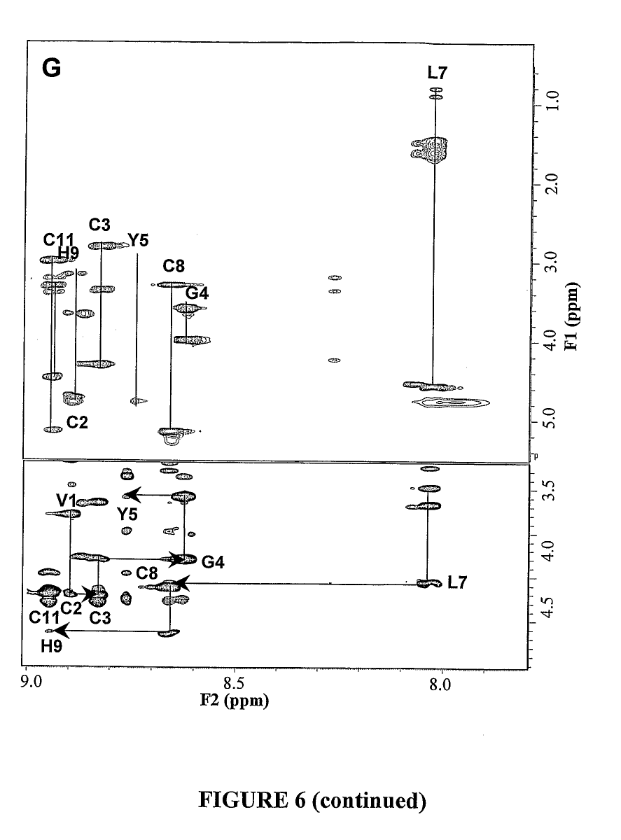

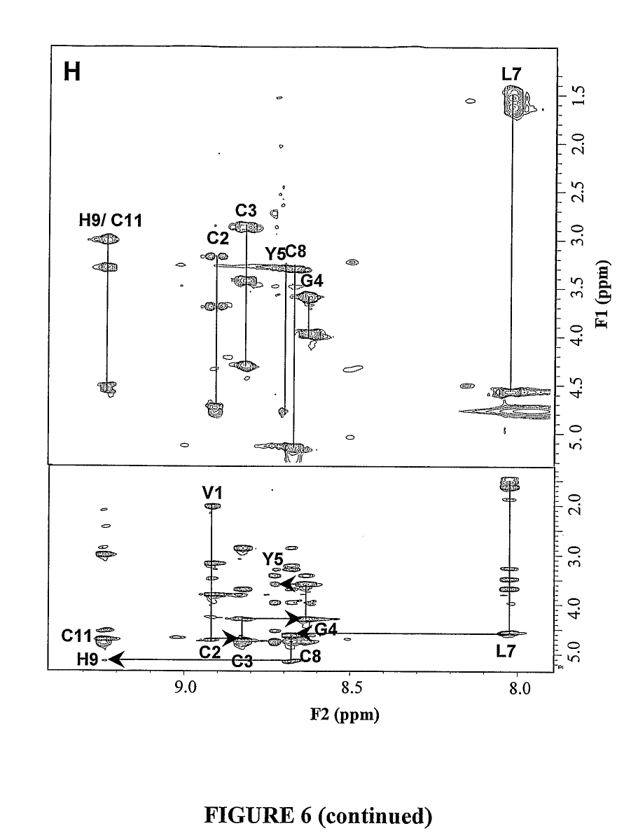

ImI Acid variant was designed to identify the role of the conserved C-terminal amidation that is seen in nearly all of the known α-conotoxin. By converting the peptide amide into the peptide acid form, we have successfully shifted the folding tendency from ˜54% of the classical globular form seen in the oxidation studies of the synthetic ImI conotoxin, to ˜67% ribbon conformation in the ImI Acid variant (FIG. 2B, FIG. 6).¹⁸ We proceed to conduct a reciprocal folding studies on a native χ/λ-conotoxin, CMrVIA conotoxin. Reciprocal studies on the effect of C-terminal amidation in CMrvIA conotoxin also resulted in a shift of structural conformation towards the globular form. However, this shift is of a much lower extent as compared to ImI Conotoxins. This is likely to be due to the presence of a confounding variable of differing second intercysteine loop size between the two classes of conotoxin.

Another structural feature examined in this work involves the replacement of the Proline residue with a Lysine residue. Lysine was selected as a substitute due to its occurance in all 3 members of the χ/λ-conotoxins at the same position of the 1^{st} intercysteine loop. Such substitution also resulted in a shift from the globular conformation in ImI conotoxin to ˜68% ribbon conformation in the P6K Amide variant.

Reciprocal studies involving CMrVIA χ/λ-conotoxin was also conducted. Native CMrVIA χ/λ-conotoxin folds to 53% ribbon conformation. When Lys6 was replaced with a Proline residue, the synthetic variant shifts to fold preferentially to 83% globular conformation. These results reinforced the point that the conserved Pro6 in ImI conotoxin has a role in determining the final conformation of the peptide toxin. Similarity in the degree of shift seen from the P6K Amide variant and ImI Acid variant suggests that both C-terminal amidation and Proline at the 6^{th} position are likely to have similar effects on the folding tendency of α-conotoxin ImI in the in vitro setting.

A further modification combining both the structural switches as seen in P6K Acid variant resulted in a further shift of folding tendency to ˜76% ribbon conformation, suggesting a likely synergistic or additive effect of the 1^{st} intercysteine loop Proline, and C-terminal amidation on the folding tendency.

Though the two structural features identified were not able to result in an absolute shift of the folding tendency from the native globular conformation to the ribbon conformation, they no doubt play a crucial role as conformational switches in the ImI conotoxin. Though the peptides fold into different predominant isoforms, the excess of one form over the other is not drastically different, suggesting that folding may occur by independent pathways. It is not clear whether proline cis-trans isomerization or hydrogen bond interactions contribute to these folding pathways.

These findings will have a significant effect in the manipulation of these small, compact peptide toxins in the development of peptide scaffolds.

The folding (oxidation) of the peptides will result in 3 possible conformations, depending on how the 4 cysteine residues pair up to form the disulfide bridges (Imagine pairing combination of [1-2, 3-4], [1-3, 2-4], [1-4, 2-3]). Based on the pairing, the peptide will adopt different shapes (that's why they are termed “globular”, “ribbon” or “beaded”), and the type of residues they will present on the surface will be different even though they have the exact sequence. The idea of using this as a scaffold is that depending on the type of pairing (and consequently the conformation resulting), the same framework can have more than 1 conformation.

### EXAMPLE 1

### Use as a Host Sequence

The sequence can be used as a rigid structural framework, in which we can insert a short segment of bioactive peptide sequence. This inserted segment can then make use of the conformation dictated by the structural scaffold so as to attain the desired activity. We have tested the sequence by inserting a well-studied tripeptide sequence (Arg-Gly-Asp) into the conotoxin framework, and the RGD-Conotoxin chimeric peptide exhibits the antiplatelet activity that we would expect of the tripeptide sequence.

Whole blood samples were freshly drawn from healthy volunteers, Platelet aggregation was measured via change in electrical impedance. Collagen (2 μg/ml)or ADP (20 μM) was used as agonist

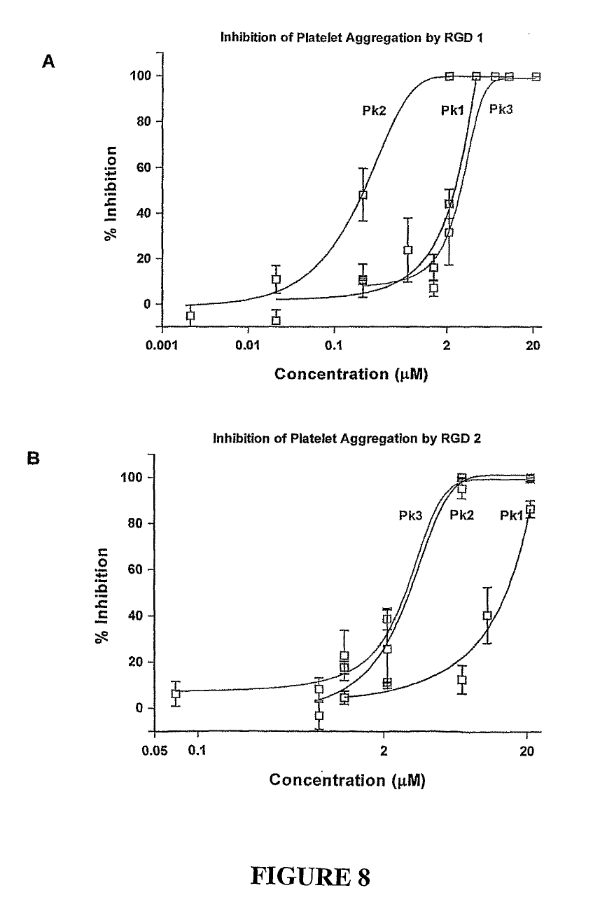

Table 5 and FIG. 8 show an antiplatelet activity assay when RGDW is put into the host sequence in intercystine loop 1 (RGD1) and intercystine loop 2 (RGD2) showing the inhibition concentration.

In several examples seen in natural protein molecules, a large percentage of the protein molecule is involved in defining the conformation of the active segment which is responsible for the biological activity. However, this active segment is usually made up of just a short length of amino acid sequence. Conventionally, there will be the effort to minimize the protein size so as to exploit the feasibility of using the active segment as a viable therapeutic agent and/or to insert into a protein scaffold so as to restrict the flexibility of the active segment into the desired conformation. A larger protein molecule will also present with problems of antigenicity due to the presence of several antigen presenting sites on parts of the molecule not relevant to the activity of interest.

Short, linear synthetic peptides corresponding to the active segments of the parent protein molecule usually will present the problem of excessive flexibility and the related high entropic cost of binding, or that the segment will be degraded easily due to the lack of a compact structure. By inserting into a scaffold that is stabilized with several restraining disulfide bridges, these problems can be reduced.

Further, by using a scaffold that is of a small size, we can rapidly and easily mass produce the peptide using chemical synthesis, as well as to easily attain the structural information using physical techniques such as NMR.

### REFERENCE LIST

(1) Gehrmann J, Alewood P F, Craik D J. Structure determination of the three disulfide bond isomers of alpha-conotoxin GI: a model for the role of disulfide bonds in structural stability. J Mol Biol. 1998;278:401-415.
(2) Balaji R A, Ohtake A, Sato K et al. lambda-conotoxins, a new family of conotoxins with unique disulfide pattern and protein folding. Isolation and characterization from the venom of Conus marmoreus. J Biol Chem. 2000;275:39516-39522.
(3) Broxton N M, Down J G, Gehrmann J et al. Alpha-conotoxin ImI inhibits the alpha-bungarotoxin-resistant nicotinic response in bovine adrenal chromaffin cells. J Neurochem. 1999;72:1656-1662.
(4) Ellison M, McIntosh J M, Olivera B M. Alpha-conotoxins ImI and ImII. Similar alpha 7 nicotinic receptor antagonists act at different sites. J Biol Chem. 2003;278:757-764.
(5) Gehrmann J, Daly N L, Alewood P F, Craik D J. Solution structure of alpha-conotoxin ImI by 1H nuclear magnetic resonance. J Med Chem. 1999;42:2364-2372.
(6) Gouda H, Hirono S. Solution structure of alpha-conotoxin ImI determined by two-dimensional NMR spectroscopy. Biochim Biophys Acta. 1999;1431:384-394.
(7) Johnson D S, Martinez J, Elgoyhen A B, Heinemann S F, McIntosh J M. alpha-Conotoxin ImI exhibits subtype-specific nicotinic acetylcholine receptor blockade: preferential inhibition of homomeric alpha 7 and alpha 9 receptors. Mol Pharmacol. 1995;48:194-199.
(8) Lamthanh H, Jegou-Matheron C, Servent D, Menez A, Lancelin J M. Minimal conformation of the alpha-conotoxin ImI for the alpha7 neuronal nicotinic acetylcholine receptor recognition: correlated CD, NMR and binding studies. FEBS Lett. 1999;454:293-298.
(9) Maslennikov IV, Shenkarev Z O, Zhmak M N et al. NMR spatial structure of alpha-conotoxin ImI reveals a common scaffold in snail and snake toxins recognizing neuronal nicotinic acetylcholine receptors. FEBS Lett. 1999;444:275-280.
(10) McIntosh J M, Yoshikami D, Mahe E et al. A nicotinic acetylcholine receptor ligand of unique specificity, alpha-conotoxin ImI. J Biol Chem. 1994;269:16733-16739.
(11) Nielsen J S, Buczek P, Bulaj G. Cosolvent-assisted oxidative folding of a bicyclic alpha-conotoxin ImI. J Pept Sci. 2004;10:249-256.
(12) Quiram P A, Sine S M. Structural elements in alpha-conotoxin ImI essential for binding to neuronal alpha7 receptors. J Biol Chem. 1998;273:11007-11011.
(13) Quiram P A, Sine S M. Identification of residues in the neuronal alpha7 acetylcholine receptor that confer selectivity for conotoxin ImI. J Biol Chem. 1998;273:11001-11006.
(14) Quiram P A, Jones J J, Sine S M. Pairwise interactions between neuronal alpha7 acetylcholine receptors and alpha-conotoxin ImI. J Biol Chem. 1999;274:19517-19524.
(15) Rogers J P, Luginbuhl P, Shen G S et al. NMR solution structure of alpha-conotoxin ImI and comparison to other conotoxins specific for neuronal nicotinic acetylcholine receptors. Biochemistry. 1999;38:3874-3882.
(16) Utkin Y N, Zhmak M N, Methfessel C, Tsetlin V I. Aromatic substitutions in alpha-conotoxin ImI. Synthesis of iodinated photoactivatable derivative. Toxicon. 1999;37:1683-1695.
(17) Wuthrich K. NMR of Proteins and Nucleic Acids. 1986.
(18) Kang T S, Vivekanandan S, Jois S D S, Kini R M. Effect of C-Terminal Amidation in Folding and Disulfide-Pairing of α-Conotoxin ImI. Angew. Chem. Int. Ed. Engl. In Press. 2005.
(19) Loughnan M, Bond T, Atkins A et al. alpha-conotoxin EpI, a novel sulfated peptide from Conus episcopatus that selectively targets neuronal nicotinic acetylcholine receptors. J Biol Chem. 1998;273:15667-15674.
(20) Fainzilber M, Hasson A, Oren R et al. New mollusc-specific alpha-conotoxins block Aplysia neuronal acetylcholine receptors. Biochemistry. 1994;33:9523-9529.
(21) Cartier G E, Yoshikami D, Gray W R et al. A new alpha-conotoxin which targets alpha3beta2 nicotinic acetylcholine receptors. J Biol Chem. 1996;271:7522-7528.
(22) Martinez J S, Olivera B M, Gray W R et al. alpha-Conotoxin EI, a new nicotinic acetylcholine receptor antagonist with novel selectivity. Biochemistry. 1995;34:14519-14526.
(23) Luo S, Kulak J M, Cartier G E et al. alpha-conotoxin AuIB selectively blocks alpha3 beta4 nicotinic acetylcholine receptors and nicotine-evoked norepinephrine release. J Neurosci. 1998;18:8571-8579.
(24) McIntosh J M, Dowell C, Watkins M et al. Alpha-conotoxin GIC from Conus geographus, a novel peptide antagonist of nicotinic acetylcholine receptors. J Biol Chem. 2002;277:33610-33615.
(25) Nicke A, Loughnan M L, Millard E L et al. Isolation, structure, and activity of GID, a novel alpha 4/7-conotoxin with an extended N-terminal sequence. J Biol Chem. 2003;278:3137-3144.
(26) Loughnan M L, Nicke A, Jones A et al. Chemical and functional identification and characterization of novel sulfated alpha-conotoxins from the cone snail Conus anemone. J Med Chem. 2004;47:1234-1241.
(27) Sandall D W, Satkunanathan N, Keays D A et al. A novel alpha-conotoxin identified by gene sequencing is active in suppressing the vascular response to selective stimulation of sensory nerves in vivo. Biochemistry. 2003;42:6904-6911.
(28) McIntosh M, Cruz L J, Hunkapiller M W, Gray W R, Olivera B M. Isolation and structure of a peptide toxin from the marine snail Conus magus. Arch Biochem Biophys. 1982;218:329-334.
(29) Gray W R, Luque A, Olivera B M, Barrett J, Cruz L J. Peptide toxins from Conus geographus venom. J Biol Chem. 1981;256:4734-4740.
(30) Zafaralla G C, Ramilo C, Gray W R et al. Phylogenetic specificity of cholinergic ligands: alpha-conotoxin SI. Biochemistry. 1988;27:7102-7105.
(31) McIntosh J M, Corpuz G O, Layer R T et al. Isolation and characterization of a novel conus peptide with apparent antinociceptive activity. J Biol Chem. 2000;275:32391-32397.

(32) Sharpe I A, Gehrmann J, Loughnan M L et al. Two new classes of conopeptides inhibit the alphal-adrenoceptor and noradrenaline transporter. Nat Neurosci. 2001;4:902-907.

TABLE 1

| | | | | | |
|---|---|---|---|---|---|
| | | | Globular | Ribbon | |
| Name | Sequence [SEQ ID NO] | Prey | m/n | m/n | Ref |

| | | | | | | |
|---|---|---|---|---|---|---|
| 4/7 Class | | | | | | |
| EpI | GCCSDPRCNMNNPDYC* | [9] | Mollusks | 5/12 | 13/4 | ¹⁹18 |
| PnIA | GCCSLPPCAANNPDYC* | [10] | Mollusks | 5/12 | 13/4 | ²⁰19 |
| PnIB | GCCSLPPCALSNPDYC* | [11] | Mollusks | 5/12 | 13/4 | ²⁰19 |
| MII | GCCSNPVCHLEHSNLC* | [12] | Fish | 5/12 | 13/4 | ²¹20 |
| EI | RDOCCYHPTCNMSNPQIC* | [13] | Fish | 5/12 | 13/4 | ²²21 |
| AUIA | GCCSYPPCFATNSDYC* | [14] | Mollusks | 5/12 | 13/4 | ²³22 |
| AUIC | GCCSYPPCFATNSGYC* | [15] | Mollusks | 5/12 | 13/4 | ²³22 |
| GIC | GCCSHPACAGNNQHIC* | [16] | Fish | 5/12 | 13/4 | ²⁴23 |
| GID | IRDγCCSNPACRVNNOHVC | [17] | Fish | 5/12 | 13/4 | ²⁵24 |
| AnIB | GGCCSHPACAANNQDYC* | [18] | Worm | 5/12 | 13/4 | ²⁶25 |
| AUIB | GCCSYPPCFATNPD C* | [19] | Mollusks | 5/11 | 12/4 | ²³22 |
| Vc1.1 | GCCSDPRCNYDHEI C* | [20] | Mollusks | 5/11 | 12/4 | ²⁷26 |
| ImI | GCCSDPRCAWR C* | [1] | Worm | 5/8 | 9/4 | 10 |
| ImII | ACCSDRRCRWR C* | [21] | Worm | 5/8 | 9/4 | 4 |
| 3/5 Class | | | | | | |
| MI | GRCCHPA CGKNYS C* | [22] | Fish | 4/9 | 10/3 | ²⁸27 |
| GI | ECCNPA CGRHYS C* | [23] | Fish | 4/9 | 10/3 | ²⁹28 |
| GIA | ECCNPA CGRHYS CGK* | [24] | Fish | 4/9 | 10/3 | ²⁹28 |
| GII | ECCNPA CGKHFS C* | [25] | Fish | 4/9 | 10/3 | ²⁹28 |
| SI | ICCNPA CGPKYS C* | [26] | Fish | 4/9 | 10/3 | ³⁰29 |
| χ/λ | | | | | | |
| CMrVIA | VCCGYKLCHO C | [27] | Mollusks | 5/7 | 8/4 | 2 |
| CMrX | GICCGVSFCYO C | [28] | Mollusks | 5/7 | 8/4 | 2 |
| MrIA | NGVCCGYKLCHO C | [29] | Mollusks | 5/7 | 8/4 | ^{31,32}3 |
| | | | | | | 1 |

TABLE 2

| | Globular | Ribbon | Beaded |
|---|---|---|---|
| ImI Acid | 30.09 ± 0.61 | 67.24 ± 0.36 | 2.67 ± 0.46 |
| P6K Acid | 19.75 ± 0.29 | 76.16 ± 0.44 | 4.08 ± 0.54 |
| ImI Cntx | 54.02 ± 0.39 | 42.97 ± 0.95 | 3.02 ± 0.56 |
| P6K Amide | 68.50 ± 1.69 | 30.23 ± 1.47 | 1.27 ± 0.39 |
| CMrVIA Cntx | 31.27 ± 0.87 | 52.92 ± 0.34 | 15.81 ± 0.54 |
| CMrVIA Amide | 33.95 ± 0.55 | 48.84 ± 0.97 | 17.22 ± 0.42 |
| CMrVIA K6P Acid | 82.94 ± 0.40 | 3.48 ± 0.26 | 13.57 ± 0.40 |
| CMrVIA K6P Amide | 93.14 ± 0.67 | 5.33 ± 0.70 | 1.53 ± 0.06 |

TABLE 3

| | Theoretical | Observed | Oxidized |
|---|---|---|---|
| | Mass (Da) | Mass (Da) | Mass (Da) |
| ImI Acid | 1356.54 | 1356.18 | 1352.16 |
| P6K Acid | 1387.65 | 1386.54 ± 0.66 | 1382.85 ± 0.48 |
| ImI Cntx | 1355.52 | 1355.85 ± 0.04 | 1350.90 ± 0.17 |
| P6K Amide | 1386.67 | 1386.45 ± 0.80 | 1381.95 ± 0.38 |
| CMrVIA Acid | 1241.57 | 1241.20 ± 0.18 | 1236.90 ± 0.17 |
| CMrVIA Amide | 1240.59 | 1240.20 ± 0.26 | 1236.30 ± 0.68 |
| CMrVIA K6P Acid | 1271.61 | 1209.70 ± 0.10 | 1205.70 ± 0.18 |
| CMrVIA K6P Amide | 1270.63 | 1208.80 ± 0.04 | 1204.96 ± 0.39 |

TABLE 4

| | | | | |
|---|---|---|---|---|
| NH | αH | βH | γH | δH |

(A)
ImI Acid Peak 1 2D NMR Chemical Shifts

| | | | | | |
|---|---|---|---|---|---|
| G1 | — | 3.775 | — | — | — |
| C2 | 8.304 | 4.507 | 2.755/2.240 | — | — |
| C3 | 8.952 | 4.927 | 3.557/3.444 | — | — |
| S4 | 8.722 | 4.692 | 3.915 | — | — |
| D5 | 7.675 | 4.941 | 2.756/2.802 | — | — |
| P6 | — | 4.348 | 2.415 | 2.051/1.956 | 3.897/3.767 |
| R7 | 8.641 | 4.259 | 1.921/1.830 | 1.648 | 3.204 |
| C8 | 8.041 | 4.410 | 3.371/3.189 | — | — |
| A9 | 8.558 | 4.145 | 1.345 | — | — |
| W10 | 7.949 | 4.957 | 3.378/3.128 | — | — |
| R11 | 8.488 | 4.972 | 1.883/1.762 | 1.641 | 3.234 |
| C12 | 7.861 | 4.365 | 3.136/2.984 | — | — |

(B)
P6K Acid Peak 2 2D NMR Chemical Shifts

| | | | | | |
|---|---|---|---|---|---|
| G1 | — | 3.830 | — | — | — |
| C2 | 8.529 | 4.682 | 2.783/3.007 | — | — |
| C3 | 8.694 | 5.032 | 3.303 | — | — |
| S4 | 8.729 | 4.583 | 3.916 | — | — |
| D5 | 8.164 | 4.712 | 2.990 | — | — |
| K6 | 8.322 | 4.088 | 1.949/1.711 | 1.466 | — |
| R7 | 7.914 | 4.409 | 1.917/1.809 | 1.623 | 3.217 |
| C8 | 8.061 | 4.775 | 3.080 | — | — |
| A9 | 8.316 | 4.279 | 1.360 | — | — |
| W10 | 7.713 | 4.840 | 3.389/3.260 | — | — |
| R11 | 8.177 | 4.595 | 1.838/1.716 | 1.580 | 3.174 |
| C12 | 8.189 | 4.595 | 3.281/3.052 | — | — |

(C)
ImI Conotoxin Peak 3 2D NMR Chemical Shifts

| | | | | | |
|---|---|---|---|---|---|
| G1 | — | 3.892 | — | — | — |
| C2 | 8.779 | 4.688 | 3.321/2.815 | — | — |
| C3 | 8.348 | 4.414 | 2.870/3.375 | — | — |
| S4 | 7.965 | 4.537 | 4.004/3.895 | — | — |
| D5 | 7.979 | 5.153 | 3.198/2.706 | — | — |
| P6 | — | 4.343 | 1.989 | 1.838/1.729 | — |
| R7 | 8.368 | 4.332 | 1.735/1.831 | 1.967 | 3.252 |
| C8 | 8.081 | 4.414 | 3.649/3.143 | — | — |
| A9 | 8.150 | 4.141 | 1.407 | — | — |
| W10 | 7.774 | 4.510 | 3.444/3.239 | — | — |
| R11 | 7.685 | 3.854 | 0.614 | 1.407 | 2.924 |
| C12 | 7.951 | 4.551 | 3.498/3.143 | — | — |

(D)
P6K Amide Peak 1 2D NMR Chemical Shifts

| | | | | | |
|---|---|---|---|---|---|
| G1 | — | 3.587/3.654 | — | — | — |
| C2 | 8.321 | 4.471 | 2.433/2.791 | — | — |
| C3 | 8.647 | 4.866 | 3.280/3.239 | — | — |
| S4 | 8.669 | 4.461 | 3.776 | — | — |
| D5 | 7.801 | 4.551 | 2.683/2.762 | — | — |
| K6 | 8.245 | 3.961 | 1.564/1.315 | 2.877 | 1.786 |
| R7 | 8.069 | 4.195 | 1.489/1.709 | 1.773 | 3.072 |
| C8 | 7.809 | 4.584 | 3.055 | — | — |
| A9 | 8.252 | 4.076 | 1.205 | — | — |
| W10 | 7.714 | 4.708 | 3.074/3.226 | — | — |
| R11 | 8.147 | 4.477 | 1.386/1.561 | 1.654 | 3.028 |
| C12 | 8.132 | 4.427 | 3.097/2.759 | — | — |

TABLE 5

| | | |
|---|---|---|
| Inhibitor | IC₅₀ (Collagen) | IC₅₀ (ADP) |

| | | | |
|---|---|---|---|
| RGD1 Pk1 | 1.48 | μM | 2.63 μM |
| RGD1 Pk2 | 0.82 | μM | 0.22 μM |
| RGD1 Pk3 | 1.64 | μM | 2.40 μM |
| RGD2 Pk1 | >15 | μM | 11.4 μM |
| RGD2 Pk2 | >15 | μM | 3.05 μM |
| RGD2 Pk3 | >15 | μM | 2.70 μM |
| Eptifibatide | 0.083 | μM | 0.023 μM |

What is claimed is:

1. A method of preparing a biologically active peptide, comprising:
incorporating a bioactive-peptide sequence into a peptide scaffold, the peptide scaffold comprising the sequence of Formula I, the bioaotive peptide sequence being incorporated into the region defined by Xₘ or the region defined by Xₙ of Formula I, wherein:
Formula I is -Cys1-Cys2-Xₘ-Cys3-Xₘ-Cys4;
Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3;
X is any amino acid;
m and n are the same or different and each is equal to or greater than 1;
and the peptide scaffold has a C-terminal group that is either of a carboxy group or an amide group; and
one or both of the following:
(i) introducing a proline residue two positions N-terminal to Cys3 or removing an existing proline residue that is two positions N-terminal to Cys3; and
(ii) converting the C-terminal group to the other of the carboxy group or the amide group;
to maintain the bioactivity of the bioactive peptide.

2. The method of claim 1, wherein the bioactive peptide sequence comprises the sequence RGD.

3. The method of claim 1 or claim 2, wherein the bioactive peptide sequence comprises the sequence RGDW.

4. The method of claim 2, wherein the bioactive peptide sequence consists of the sequence RGD.

5. The method of claim 3, wherein the bioactive peptide sequence consists of the sequence RGDW.

6. A method of altering the conformation of a peptide, the method comprising modifying a peptide comprising the sequence of Formula I and a C-terminal group that is either of a carboxy group or an amide group to convert the C-terminal group to the other of the carboxy group or the amide group, wherein:
Formula I is -Cys1-Cys2-Xₘ-Cys3-Xₙ-Cys4-;
Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3;
X is any amino acid; and
m and n are the same or different and each is equal to or greater than 1.

7. The method of claim 6 further comprising introducing a proline residue two positions N-terminal to Cys3 or removing a proline residue that is two positions N-terminal to Cys3.

8. A peptide comprising a conotoxin consensus sequence as defined in Formula I, and having one or more amino acid residues inserted or substituted between Cys2 and Cys3 such that the region defined by Xₘ differs from the corresponding region in any wildtype conotoxin sequence, or having one or more amino acid residues inserted or substituted between Cys3 and Cys4 such that the region defined by Xₙ differs from the corresponding region in any wildtype conotoxin sequence, wherein:
Formula I is -Cys1-Cys2-Xₘ-Cys3-Xₙ-Cys4-;
Cys1, Cys2, Cys3 and Cys4 are cysteine residues that together form two disulfide bonds, between Cys1 and Cys3 and between Cys2 and Cys4, between Cys1 and Cys2 and between Cys3 and Cys4, or between Cys1 and Cys4 and between Cys2 and Cys3;
X is any amino acid; and
m and n are the same or different and each is equal to or greater than 1;
and wherein the peptide does not have a proline residue two positions N-terminal to Cys3 and has a C-terminal carboxy group, the peptide having the tendency to adopt a ribbon conformation.

9. The peptide of claim 8 wherein the amino acid sequence RGD is inserted between Cys2 and Cys3 or between Cys3 and Cys4.

10. The peptide of claim 8 wherein the amino acid sequence RGD is inserted between Cys2 and Cys3 or between Cys3 and Cys4.

11. A peptide comprising the sequence as set forth in any one of SEQ ID NOS. 3, 4, 7 or 8.

12. A peptide consisting of the sequence as set forth in any one of SEQ ID NOS. 3, 4, 6, 7 or 8.

13. A biologically active peptide comprising a peptide scaffold and a bioactive peptide sequence, the peptide scaffold comprising the sequence of Formula I, the bioactive peptide sequence being incorporated in to the region defined by Xₘ,or the region defined by Xₙ of Formula I, wherein:
Formula I is Cys1-Cys2-Xₘ-Cys3-Xₙ-Cys4-;
Cys1, Cys2, Cys3 and Cys4 are cysteine residues that together form two disulfide bonds, between Cys1 and Cys3 and between Cys2 and Cys4,
between Cys1 and Cys2 and between Cys3 and Cys4, or between Cys1 and Cys4 and between Cys2 and Cys3;
X is any amino acid;
m and n are the same or different and each is equal to or greater than 1; and
in which a proline residue normally occurring in the peptide scaffold sequence two positions N-terminal to Cys3 has been removed.

14. The peptide of claim 13 having a C-terminal group that is either of a carboxy group or an amide group.

15. The peptide of claim 13 or claim 14 wherein the amino acid sequence RGD is inserted between Cys2 and Cys3 or between Cys3 and Cys4.

16. The peptide of claim 13 or claim 14 wherein the amino acid sequence RGDW is inserted between Cys2 and Cys3 or between Cys3 and Cys4.
